(19) United States
(12) Plant Patent Application Publication
(10) Pub. No.: US 2008/0127379 P1
(43) Pub. Date: May 29, 2008

(54) Mandarin plant named Merbeingold 2350

(76) Inventor: Stephen Richard Sykes, Mildura (AU)

Correspondence Address: COOPER & DUNHAM, LLP, 1185 AVENUE OF THE AMERICAS, NEW YORK, NY, 10036, US

(21) Appl. No.: 11/729,049

(22) Filed: Mar. 27, 2007

(30) Foreign Application Priority Data
Oct. 16, 2006 (AU) 2006/278 (PBR)

(51) Int. Cl. A01H 5/00

(52) U.S. Cl. PLT/202

(57) ABSTRACT

Merbeingold 2350 is a new Australian variety of mandarin. It was selected from the progeny obtained following a controlled pollination of Imperial mandarin×Ellendale tangor in 1984. It was selected because it is strongly parthenocarpic and is capable of yielding seedless fruits, which are sweet, juicy and easy-to-peel. Fruit maturity is mid season in the Murray Valley of Australia. The rind of Merbeingold is thin, orange-to-orange red, strong yet easily peeled. Its fruits can be snap picked, eliminating a need to clip. Internally, the flesh of Merbeingold 2350 is attractive, orange in colour and juicy with a good sugar/acid balance.

This application claims priority of Australian Plant Breeder's Right Application No. 2006/278, filed Oct. 16, 2006, the contents of which are incorporated by reference herein.

Merbeingold 2350 was accepted for Plant Breeder's Right (“PBR”) registration on Dec. 1, 2006 when a provisional PBR was granted. Acceptance was published in the Plant Varieties Journal (2007) Vol. 19, No 4, p23 (see, ipaustralia.gov.au/pdfs/plantbreed/PVJ_{—}19_{—}4.pdf). The Commonwealth of Australia Plant Breeder's Rights Act 1994 follows the guidelines of the International Convention for the Protection of New Varieties of Plants—UPOV Convention (1961), as revised at Geneva (1972, 1978 and 1991). Australia is a UPOV Member State.

### LATIN NAME OF THE GENUS AND SPECIES OF THE PLANT CLAIMED

Citrus reticulata×(C. reticulata×C. sinensis)

### VARIETY DENOMINATION

Merbeingold 2350

### BACKGROUND OF THE INVENTION

Merbeingold 2350 is a new variety of mandarin (Citrus species) bred by CSIRO Plant Industry. A copending application has been made for the variety Merbeingold 2336, which is a sibling selected from the same family as Merbeingold 2350.

Merbeingold 2350 is a mandarin variety selected from a family produced by making a controlled cross between Imperial mandarin (seed parent) with Ellendale tangor (pollen parent). Thus, the botanical name for the plant is:
Citrus reticulata×(C. reticulata×C. sinensis)

The plant may be used for horticultural production of mandarin fruits.

Merbeingold 2350 was selected from a family of 241 hybrids generated by a controlled cross-pollination of Imperial mandarin (maternal parent) with Ellendale tangor (pollen parent).

Imperial mandarin is an Australian variety that originated at Emu Plains, NSW, as a chance seedling in 1890. It is possibly a hybrid of the Mediterranean mandarin. Imperial trees are vigorous, upright and of medium size and they yield fruits that are early maturing.

Ellendale tangor is another Australian variety that was discovered as a chance seedling at Burrum, Queensland in 1878. Although its parentage is unknown, its characteristics and fruit size indicate that it is a tangor (mandarin×orange cross). Ellendale trees are generally large and of a spreading-round habit and produce large mid-to-late season fruit depending on where they are grown.

The seediness of fruits from both Imperial and Ellendale can be variable ranging from many-to-few-to-zero depending on the proximity of other sources of pollen. Both varieties are capable of producing fruits parthenocarpically.

CSIRO crossed Imperial with Ellendale to combine the characteristics of the two varieties and generate new parthenocarpic hybrids for selection of potential new varieties of seedless mandarins.

The controlled cross-pollination was conducted by emasculating an un-opened flower bud of the maternal parent and applying pollen from the paternal parent to the receptive stigma using a sterile soft-haired paintbrush. Pollen of Ellendale tangor was collected by drying anthers, which had been removed from unopened flower buds, in Petri dishes over silica gel in a dessicator. Dried dehisced anthers were stored in sealed glass vials over silica gel at 4° C. until needed. The cross was made in 1984 and the resultant seeds were extracted from fruits in 1985 and sown in a standard seed bed under glasshouse conditions. Emergent seedlings were transferred to a standard potting mix in pots and maintained under glasshouse conditions until they were rowed out in the breeding orchard at a planting density of 2 m within and 6 m between rows. Hybrid seedlings were maintained under irrigated orchard conditions thereafter. Standard citrus cultivation techniques were used to maintain the trees including application of fertilisers.

When hybrid 2350 flowered, it was subjected to a range of pollination treatments to assess its potential for producing seedless fruits. Fruits were harvested over 4 years and assessed for fruit quality, Based on the data collected, hybrid 2350 was selected for entry into second phase evaluation trials. The selection was entered into a comparative trial at CSIRO Plant Industry Koorlong (NW Victoria). It was also entered along with other selections into regional test plots with anonymous cooperating citrus growers under confidential testing agreement arrangements to protect inherent intellectual property. Based on its performance in these trials and test plots, hybrid 2350 was named Merbeingold 2350.

Daughter trees of Merbeingold 2350 propagated from the original seedling tree by asexual or vegetative means are uniform and stable. Similarly grand-daughter trees are uniform and stable. Trees of Merbeingold 2350 have been propagated by grafting or budding to seedling rootstocks, by top-working to established orchard trees and by rooting cuttings, confirming its uniformity and stability.

### SUMMARY OF THE INVENTION

Fruits of Merbeingold 2350 have the capacity to be seedless and seed numbers are dependent on cross-pollination. In regional test plots, mean seed numbers have ranged from less than 1 up to 10 seeds per fruit. Low seed numbers have occurred where trees are planted close to navel oranges, which because they are pollen sterile, cannot cross-pollinate Merbeingold 2350's flowers. Under these circumstances, seedless and low-seeded fruits have been harvested. Where higher seed numbers have been recorded, trees of Merbeingold 2350 have been surrounded by strong pollen producers such as Valencia orange, Murcott tangor and Minneola tangelo. In these situations, cross-pollination is promoted and seedier fruits result. Clearly, successful adoption of Merbeingold 2350 to produce seedless and low-seeded fruits will depend on its location with regard to potential cross-pollinator varieties. In orchards where seed numbers have been one-or-less per fruit, a large proportion of the fruits have been seedless.

The rind is thin and the fruits are easy-to-peel leaving segments free of albedo tissue. The segments are readily separated without breaking and are tender with soft walls resulting in a pleasant texture when eaten. The rind, though thin, is strong giving a robust intact fruit suggesting it will ship well and be suited for export.

The juice is sweet with a °Brix of around 11-12. With an acid concentration of 1% or just under at maturity, fruits of Merbeingold 2350 have a pleasant sweet flavour. The juice does not have an overly distinctive flavour.

The rind is an attractive red-orange which should have high appeal in the market place. In this regard, the fruit has been compared to the variety Nova but it does not appear to suffer granulation like Nova. Fruits of Merbeingold 2350 can be snapped from the tree leaving the button intact. They reach maturity during the period late June-through-August in Australia's Murray Valley depending on soil type, rootstock and season. Fruit size is good with mean fruit weights in the range 120-140g.

### BRIEF DESCRIPTION OF THE DRAWING

FIGS. 1A-1C: Show leaves from Merbeingold 2350 (FIG. 1A) and its parents, Imperial mandarin (FIG. 1B) and Ellendale tangor (FIG. 1C). In Merbeingold 2350 the Lamina length:width ratio=2.2±0.2; in Imperial mandarin the Lamina length:width ratio=2.7±0.2; and in Ellendale tangor the Lamina length:width ratio=2.1±0.2

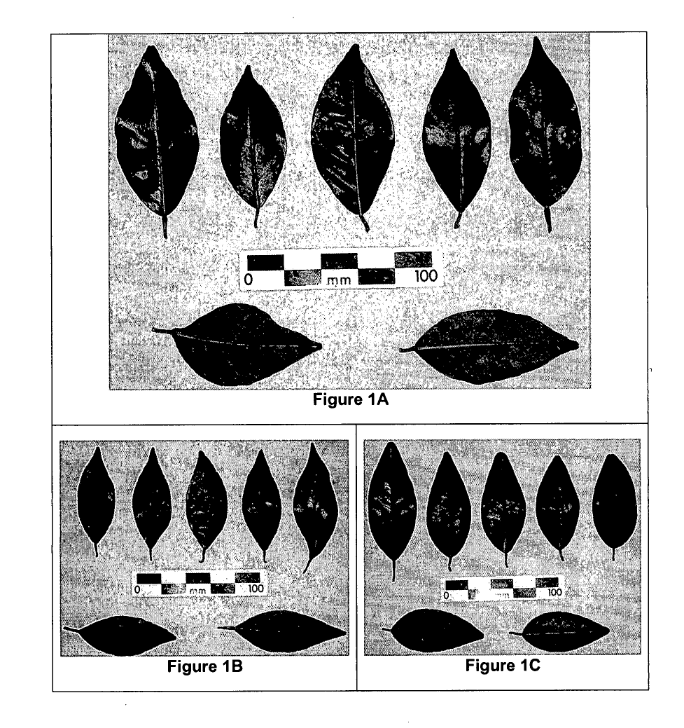

FIGS. 2A-2C: Show fruit of Merbeingold 2350 (FIG. 2A) and its parents, Imperial mandarin (FIG. 2B) and Ellendale tangor (FIG. 2C).

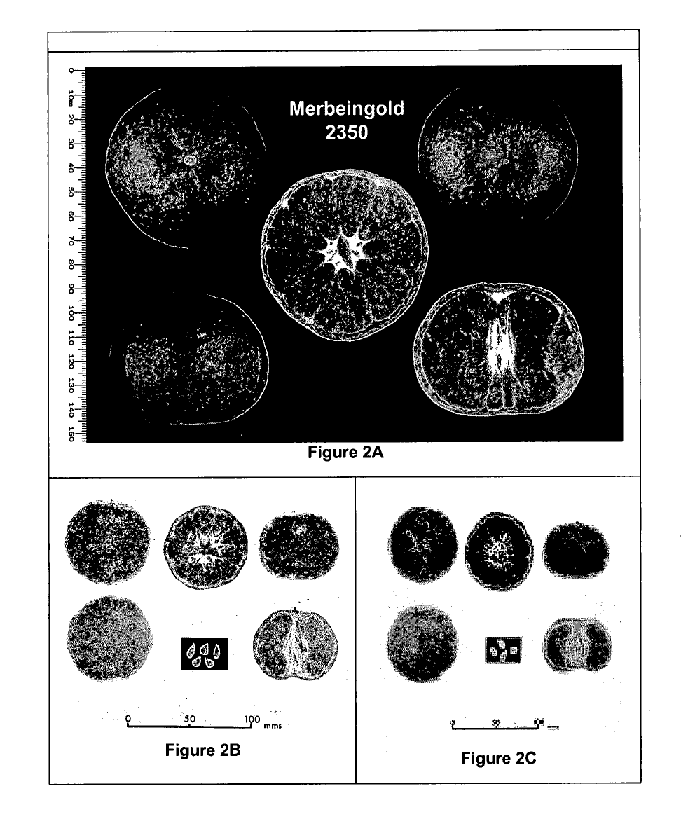

### DETAILED BOTANICAL DESCRIPTION OF THE PLANT

Variety—Merbeingold 2350

Citrus reticulate×(C. reticulata×C. sinensis)

Descriptors used herein from 1) European Union Community Plant Variety Office, Protocol for distinctness, uniformity and stability tests. Citrus L.—Group 1 MANDARINS. CPVO-TP\201\1 Adopted Nov. 11, 2004, and 2) IPGRI (1999) Descriptors for Citrus. International Plant Genetic Resources Institute, Rome, Italy (ISBN 92-9043-425-2).
Plant: Diploid, growth habit spreading-to-upright, tree shape spheroid-ellipsoid, density of branches medium, branch angle narrow, trunk surface smooth, shoot tip green and surface glabrous.
Spine (thorn): Either absent or very sparse on adult tree, length on adult tree short <5 mm, shape straight.
Leaf: Evergreen, type simple, shape ovate, intensity of green colour on lamina medium-to-dark, colour variegation absent, margin very weakly dentate-to-mostly entire, shape of apex acute, lamina length medium (85±8 mm), lamina width narrow-to-medium (39±3 mm), lamina length to width ratio 2.2±0.2, cross-sectional shape intermediate, lamina undulation absent-to-very slight, lamina attachment brevipetiolate, petiole length short (10±3 mm), petiole wings obdeltate and very narrow, petiole attachment to stem curved, junction between petiole and lamina articulate, colour of lamina upper/lower surface same.
Flower: Hermaphrodite, arrangement solitary and as a raceme, position axillary and terminal, length of petal short-to-medium, anther colour medium yellow, viable pollen sparse-to-normal, length of anther relative to style shorter (inferior), colour of open flower white, 5 petals per flower, stamens >4 per petal, style straight and complete, self-compatible.
Fruit: Maturity mid-season (late June-August, Australia), borne both inside and outside canopy, obloid, attachment to stalk medium-to-strong, broadest at equator, shape in transverse section circular, base flattened-to-slightly concave, apex flat-to-truncate, neck absent, slight depression at stalk end, number of radial grooves at stalk end intermediate, collar absent, distal part flattened, no depression at distal end, areola absent, stylar end closed, stylar scar small, style not persistent, navel opening absent, radial grooves at distal end absent, rind surface predominantly orange-to-orange red, surface glossiness strong, surface roughness medium, rind smooth to slightly pitted, oil glands all more-or-less same size, pitting of oil glands present pebbling absent, oil glands conspicuous, small and of low density, rind thin (2-3 mm), rind adherence to flesh weak-to-medium, rind strength strong, rind oiliness medium, albedo white and loose, amount of albedo adhering to flesh very small, albedo strands absent, flesh medium-to-dark orange, pulp colour uniform, pulp firmness intermediate, pulp texture fleshy, core small-to-medium, round and sparsely filled, segments uniform, rudimentary segments absent, number of well developed segments medium (5-to-10; mean 9.1±0.7), adherence of segment walls to each other medium, strength of segment walls medium, vesicle length long, vesicle thickness medium-to-thin, navel absent, juiciness high, soluble solids medium-to-high 9-15 °Brix, acidity medium, strength of fibre medium, parthenocarpy present.
Seed: Number of seeds under controlled manual self pollination few (0-4), 0-to-15 under open pollination depending, on proximity of other pollen sources, monoembryonic, shape ovoid, length short, width narrow, surface smooth, external colour whitish/cream, colour of inner seed coat light brown, chalazal cream, cotyledons white-to-light yellow-cream.

What is claimed is:

1. A new and distinct parthenocarpic mandarin variety named Merbeingold 2350 as illustrated and described.
